(19) United States
(12) Patent Application Publication
(10) Pub. No.: US 2026/0345004 A1
(43) Pub. Date: Nov. 5, 2026

(54) IMAGING OPTICAL SYSTEM AND MEASUREMENT DEVICE

(71) Applicant: CANON VIRGINIA, INC., Newport News, VA (US)

(72) Inventor: Hiroto Kano, Utsunomiya-shi (JP)

(21) Appl. No.: 19/125,438

(22) PCT Filed: Oct. 31, 2023
(86) PCT No.: PCT/US2023/036497

§ 371 Date: Apr. 29, 2025

Related U.S. Application Data
(60) Provisional application No. 63/381,729, filed on Oct. 31, 2022.

(51) Int. Cl. A61B 3/15; A61B 3/12

(52) U.S. Cl. CPC A61B 3/156; A61B 3/12

(57) ABSTRACT

In an imaging optical system that guides reflected light from the retina of an eye to an imaging device, an intermediate image conjugate to the retina and the imaging plane of the imaging device is formed, and the imaging optical system includes a first optical system, a polarization-selective diffractive optical element and an array optical system in order from the retina along an optical path direction, and the polarization state of the reflected light is separated by a polarization-selective diffractive optical element.

### RELATED APPLICATIONS AND INCORPORATION BY REFERENCE

The application claims priority to U.S. Provisional Patent Application Ser. No. 63/381,729 filed Oct. 31, 2022, which is hereby incorporated herein in its entirety by reference.

### FIELD OF THE DISCLOSURE

The present disclosure relates to an imaging optical system and a measurement device equipped with the same.

### BACKGROUND

In the human eye, the fovea (pit) exists in the center of the macula of the retina, and it is known that the best visual acuity is achieved in the fovea. It is also known that by detecting the birefringence state caused by the Henle fibers surrounding the fovea, it is possible to measure where the subject is staring (vision fixation state).

WO 99/20173 and U.S. Pat. No. 10,111,584 provide an ophthalmology nerve scanner (examination device) having a projection device that projects a projection image onto the retina of the eye and a photodetector that acquires a reflection image that shows the vision fixation state of the eye reflected by the retina. The photodetector in WO 99/20173 separates the polarization information from the light reflected by the retina by a polarization beam splitter (PBS) and acquires the respective intensities. In addition, the photodetector disclosed in U.S. Pat. No. 10,111,584 is placed in a position conjugate to the retina of the eye and photographs the light reflected from the retina of the eye as a two-dimensional image.

However, in the measuring device disclosed in the above WO 99/20173, the optical path is divided in 90-degree directions when the polarization is separated by the PBS, so it is necessary to arrange multiple sensors for each polarization. A similar configuration is required when this configuration is used in U.S. Pat. No. 10,111,584. However, in such a configuration, the arrangement becomes complicated, and it is necessary to suppress their relative misalignment and the like in order to prevent the deterioration of the detection accuracy, and high position accuracy is required. As a result, inspection equipment becomes more complex and larger.

Therefore, a compact or miniaturized inspection device with a simple configuration is desired.

### SUMMARY

The present disclosure provides a compact or miniaturized inspection device with a simple configuration.

In the present disclosure, an imaging optical system that guides reflected light from the retina of the eye to the imaging device forms an intermediate image conjugate to the retina and the imaging plane of the imaging device, and the imaging optical system includes a first optical system, a polarization-selective diffractive optical element, and an array optical system in order from the retina along the optical path direction with the polarizing diffractometer separating the polarization state of the reflected light.

According to the above configuration, a compact or miniaturized imaging optical system with a simple configuration can be provided.

These and other embodiments, objects, features, and advantages of the present disclosure will become apparent upon reading the following detailed description of exemplary embodiments of the present disclosure, when taken in conjunction with the appended drawings, and provided claims.

### BRIEF DESCRIPTION OF THE DRAWINGS

The accompanying drawings, which are incorporated herein and form part of the specification, illustrate various embodiments, objects, features, and advantages of the present disclosure.

FIG. 1 is a view showing the imaging optical system (100) on the XZ plane according to an embodiment of the disclosure.

FIG. 2 is a view showing the imaging optical system (100) on the YZ plane according to an embodiment of the disclosure.

FIG. 3A-B is a view showing the PSD element constituting the imaging optical system (100).

FIG. 4A-B is a view showing the arrangement of the array optical system of the imaging optical system (100) of an embodiment of the present disclosure.

FIG. 5 is a view showing a modification of the imaging optical system of an embodiment of the present disclosure.

FIG. 6A-B is a view showing the measurement device according to Example 1.

FIG. 7A-B is an enlarged view of the measuring device according to Example 1.

FIG. 8A-B is an enlarged view of the measuring device according to Example 2.

FIG. 9A-B is an enlarged view of the measuring device according to Example 3.

FIG. 10 is a block diagram of the measuring device in each example.

FIG. 11 is a further diagram of the measuring device of FIG. 10.

FIG. 12 is an enlarged view of the measuring device according to Example 1 where LA and LB are depicted.

Throughout the figures, the same reference numerals and characters, unless otherwise stated, are used to denote like features, elements, components or portions of the illustrated embodiments, and duplicate descriptions are omitted. Each drawing may, for the sake of convenience, be drawn with a scale different from the actual scale. Moreover, while the subject disclosure will now be described in detail with reference to the figures, it is done so in connection with the illustrative exemplary embodiments. Any components shown but not described in any of the Figures have the same description as otherwise provided in the specification. It is intended that changes and modifications can be made to the described exemplary embodiments without departing from the true scope and spirit of the subject disclosure as defined by the appended claims.

### DETAILED DESCRIPTION

The present disclosure has several embodiments and relies on patents, patent applications and other references for details known to those of the art. Therefore, when a patent, patent application, or other reference is cited or repeated herein, it should be understood that it is incorporated by reference in its entirety for all purposes as well as for the proposition that is recited.

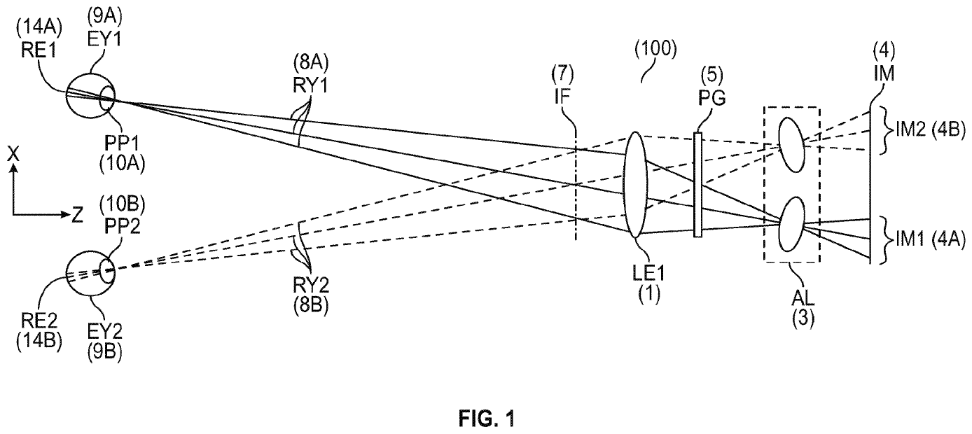

FIG. 1 is a view showing the imaging optical system (100) on the XZ plane according to a first embodiment. The imaging optical system (100) is a device that measures the vision fixation state of the eyes EY1 (9A) and EY2 (9B). The imaging optical system (100) consists of a first optical system LE1 (1), a polarization-selective diffractive optical element PG (5), and an array optical system AL (3). The imaging surface IM (4) corresponds to a light-receiving surface such as a CCD (Charge Coupled Device) sensor or a CMOS (Complementary Metal Oxide Semiconductor) sensor. The retina RE1 (14A) and RE2 (14B) of each eye EY1 (9A) and EY2 (9B) are conjugate to the intermediate image IF (7), and the intermediate image IF (7) returns the imaging optical system (100) and is conjugate to the imaging plane IM (4).

Light reflected from the retina RE1 (14A) is shown as RY1 (8A) (solid line), and light reflected from the retina RE2 (14B) is shown as RY2 (8B) (dashed line). The reflected light RY1 (8A) is once imaged at the intermediate image IF (7) position, and then imaged at the imaging position IM1 (4A) of the imaging surface IM (4) by the imaging optical system (100). In addition, the reflected light RY2 (8B) is imaged once at the intermediate image IF (7) position, and then imaged at the imaging position IM2 (4B) of the imaging surface IM (4) by the imaging optical system (100). Each optical system of the array optical system AL (3) is arranged in an approximately conjugate position with respect to the pupil (lens) PP1 (10A) of the eye EY1 (9A) and the pupil PP2 (10B) of the eye EY2 (9B), respectively. Both the first optical system LE1 (1) and the array optical system AL (3) have positive power.

Here, the first optical system LE1 (1) and the array optical system AL (3) may each be composed of multiple optical elements. A reflective device may also be employed. In addition, although the polarization-selective diffractive optical element PG (5) in FIG. 1 is composed of one sheet, multiple sheets may also be employed.

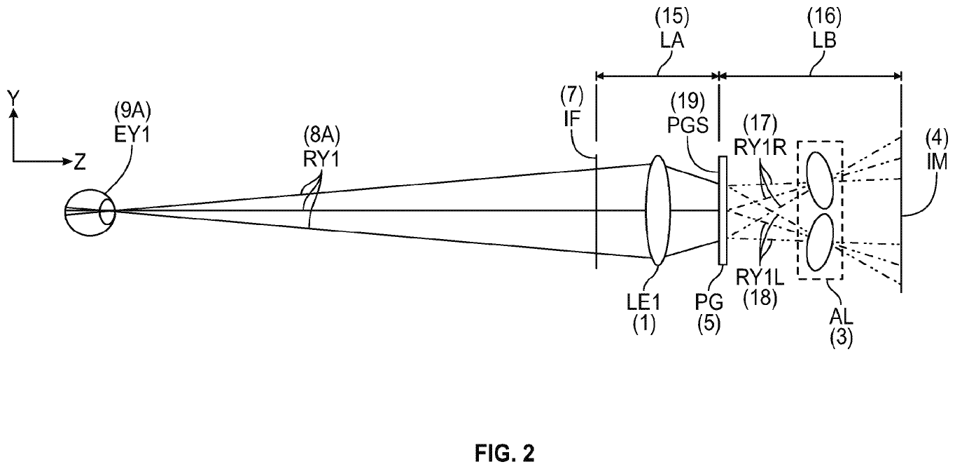

FIG. 2 shows the imaging optical system (100) on the YZ plane according to a first embodiment. The reflected light RY1 (8A) from the eye EY1 (9A) is once imaged at the intermediate image IF (7) position, and then diffracted by the diffraction plane PGS (19) of the polarization-selective diffractive optical element PG (5). Due to the polarization state of reflected light RY1 (8A), it is separated into reflected light RY1R (17) and reflected light RY1L (18), and each is imaged on the imaging surface IM (4). On the other hand, the reflected light RY2 (8B) from the eye EY2 (9B) (not shown) is also separated into two reflected lights in the same way as the reflected light RY1 (8A) due to the polarization state. The array optical system AL (3) is arranged so that the light that has separated the reflected lights RY1 (8A) and RY2 (8B) into 2 luminous fluxes respectively by the polarization-selective diffractive optical element PG (5) is guided to the imaging plane IM (4).

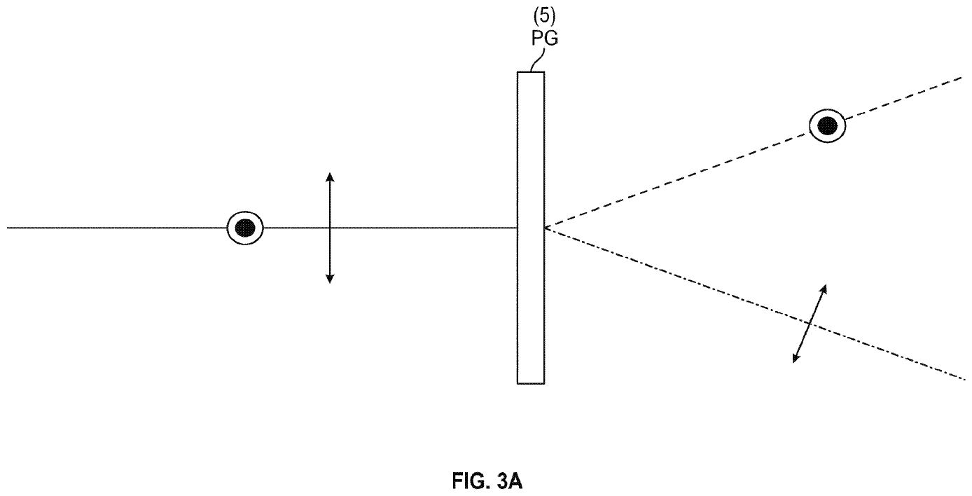

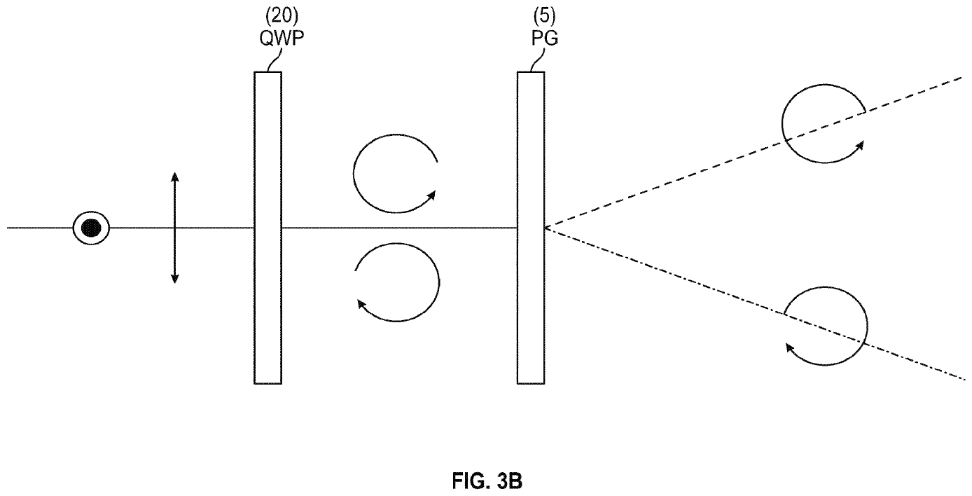

FIG. 3A-B is a view showing the polarization-selective diffractive optical element PG (Polarization Grating) (5) constituting the imaging optical system (100) of this embodiment. The polarization-selective diffractive optical element PG (5) is a device whose diffraction changes according to the rotation direction of circularly polarized light. The polarization-selective diffractive optical element PG (5) can polarize circularly polarized light with high diffraction efficiency, and diffracts unpolarized and linearly polarized light only in the ±1^{st} order. In FIG. 3A, one form of the polarization-selective diffractive optical element PG (5) is shown. The light made incident from the left in FIG. 3A is diffracted by the polarization-selective diffractive optical element PG (5), and the light is guided to the upper side for the deep-forward polarization component and to the lower side for the up-down polarization component. Since the diffraction angle of the polarization-selective diffractive optical element PG (5) is usually 20 degrees or less, each polarization makes it easier to guide the beam in the same direction compared to the polarization beam splitter PBS. Therefore, it is not necessary to arrange multiple image devices for the PBS, and size reduction of the optical system is expected. Each diffraction angle of the polarization-selective diffractive optical element PG (5) in FIG. 3A is almost identical, but differing diffraction angles are also allowed. In addition, in order to separate each polarization state, only one may be diffracted, and the other may be separated as zero-order light without being diffracted. Moreover, the diffraction direction need not be the same plane. The polarization-selective diffractive optical element PG (5) in FIG. 3B shows a device whose diffraction direction differs depending on the rotation direction of circularly polarized light. In order to separate polarization components similar to those in FIG. 3A using this polarization-selective diffractive optical element PG (5), a ¼ wavelength plate QWP (20) may be placed in front of it.

Next, the configuration of the polarization-selective diffractive optical element will be explained using FIG. 2. LA (15) indicates the interval between the intermediate image IF (7) and the diffraction plane PGS (19) of the polarization-selective diffractive optical element PG (5), and LB (16) indicates the interval between the diffraction plane PGS (19) and the imaging plane IM (4). By providing the polarization-selective diffractive optical element PG (5), it becomes possible to configure the LA (15) so that it becomes shorter. For example, it can be set as LA≤LB in the imaging optical system (100) of this embodiment. Thus, the light separated by the array optical system AL (3) can be effectively guided to the imaging plane IM (4) without increasing the diffraction angle of the polarization-selective diffractive optical element (5). This makes it possible to shorten the overall optical path length. The size reduction of the imaging optical system is also expected. However, the relationship between LA (15) and LB (16) is not limited to this, and even if LA>LB, it is possible to reduce the size of the imaging optical system compared with the case of separating the polarization by the PBS by using the polarization-selective diffractive optical element PG (5).

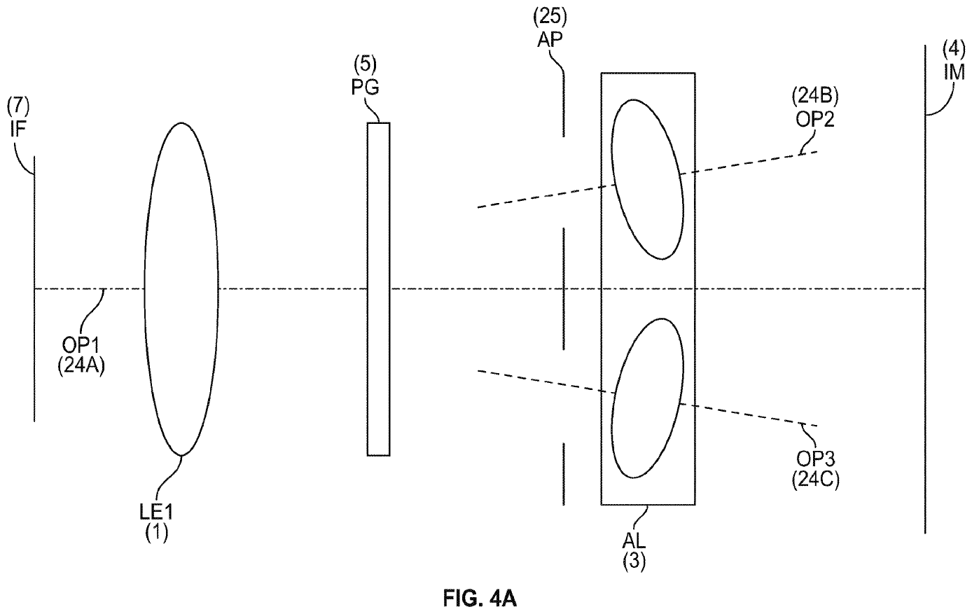

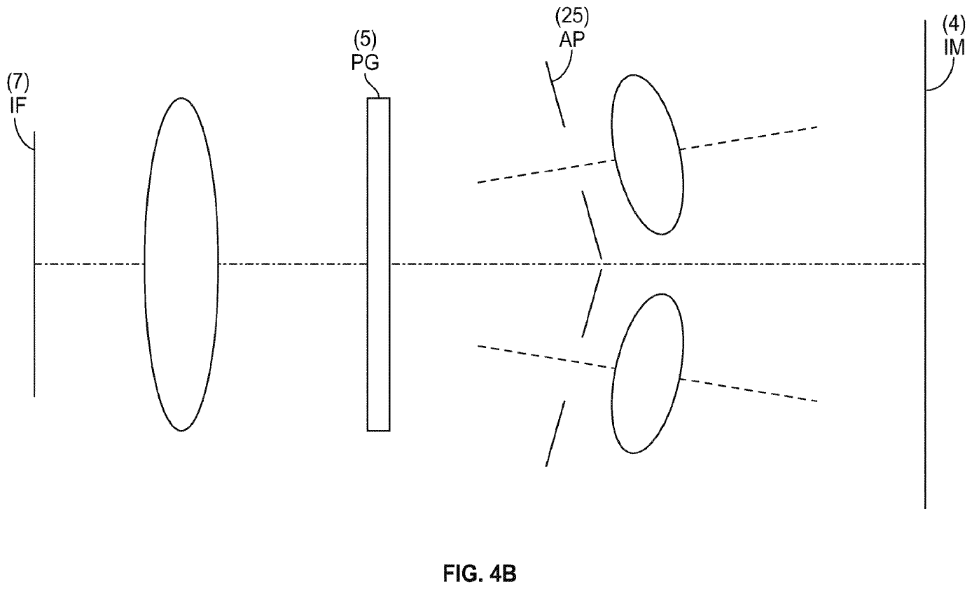

FIG. 4A-B is a view showing the arrangement of the array optical system of the imaging optical system (100) of this embodiment. In FIG. 4A, OP1 (24A) represents the optical axis composed by the first optical system LE1 (1), and OP2 (24B) and OP3 (24C) represent each optical axis of the array optical system AL (3). In FIG. 4A, the optical axes OP2 (24B) and OP3 (24C) have inclination eccentricity with respect to the optical axis OP1 (24A). Since the reflected light from both eyes, and the diffracted light after passing through the polarization-selective diffractive optical element respectively proceed with inclination with respect to the optical axis OP1 (24A), it is desirable that the optical axes OP2 (24B) and OP3 (24C) are made inclined and eccentric along it. This facilitates aberration correction and size reduction of the imaging optical system is expected. The inclination amounts of the optical axes OP2 (24B) and OP3 (24C) with respect to the optical axis OP1 (24A) may be different. Also, AP (25) indicates the light-shielding area. The light-shielding area AP (25) is arranged between the polarization-selective diffractive optical element (5) and the array optical system (3). This makes it possible to effectively prevent unwanted light generated by the polarization-selective diffractive optical element. The light-shielding area AP (25) may also serve as the aperture stop of the imaging optical system (100). Moreover, the light-shielding area AP (25) may be perpendicular to the optical axis OP1 (24A) as shown in FIG. 4A, and it may be arranged perpendicular to the optical axes OP2 (24B) and OP3 (24C) as shown in FIG. 4B.

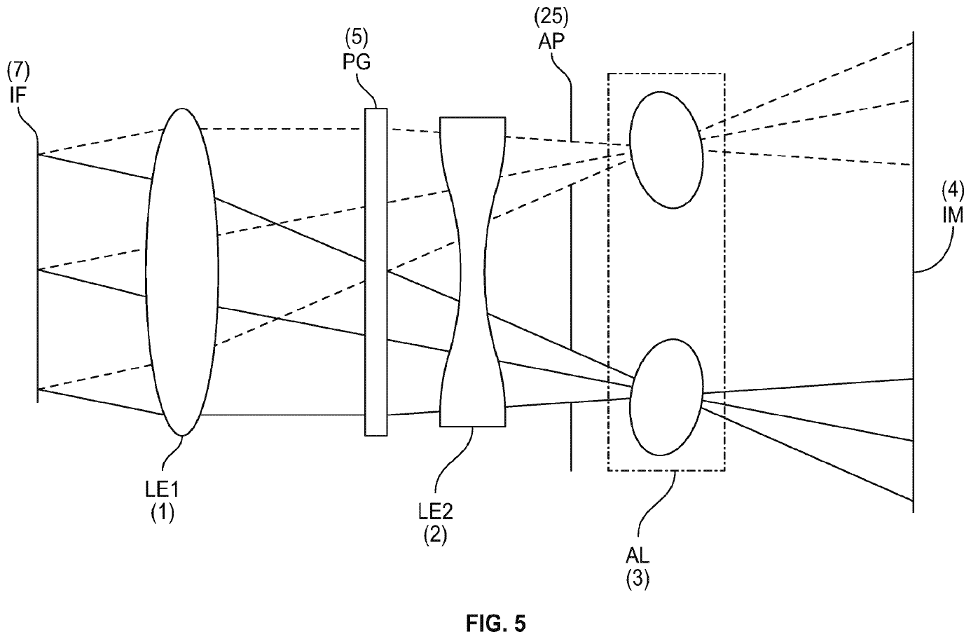

FIG. 5 is a view showing a modification of the imaging optical system of this embodiment. The imaging optical system shown in FIG. 5 provides a second optical system LE2 (2) between the polarization-selective diffractive optical element PG (5) and the light-shielding area AP (25). The first optical system (1) and the array optical system (3), have positive power (convex), and the second optical system LE2 (2) has negative power (concave). This facilitates suppressing the field curvature and shortening the optical path, and makes it possible to reduce the size of the imaging optical system (100). In addition, by arranging it after the polarization-selective diffractive optical element PG (5), the suppression of various aberrations becomes possible, since the diffraction angle of the polarization-selective diffractive optical element (5) can be reduced. The second optical system LE2 (2) may also consist of multiple optical elements.

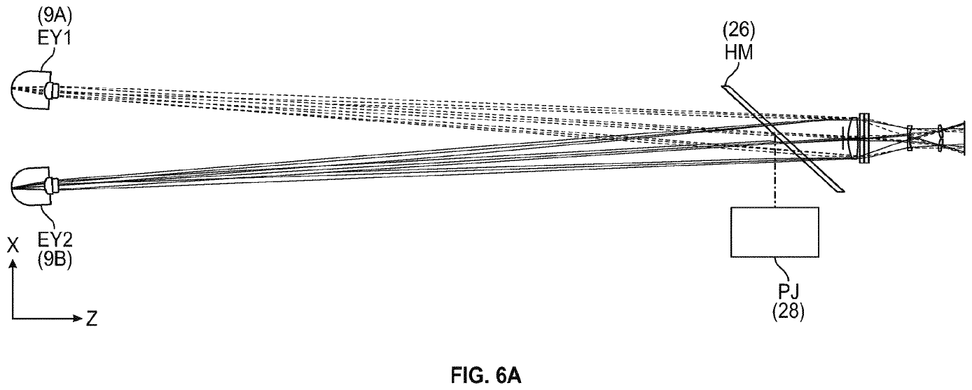

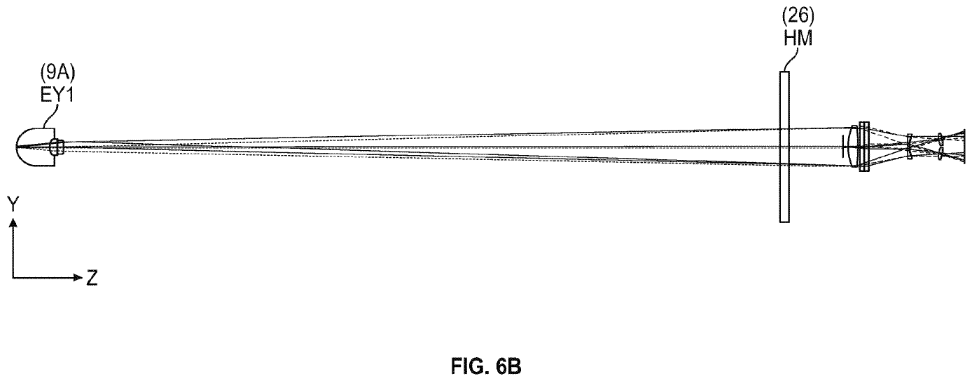

FIG. 6A-B is a view showing the measurement device according to the Example 1 of this embodiment. The light illuminated by the illuminator PJ (28) is reflected by the half-mirror HM (26) and guided to the retina of the eyes EY1 (9A) and EY2 (9B). The illumination image formed by the illuminator PJ (28) is conjugate to the retinae of the eyes EY1 (9A) and EY2 (9B). In addition, the wavelength of the light source (not shown) of the illuminator PJ (28) can be in the range of 800-900 nm from the viewpoint to be described later. Because light measures the retina through the cornea, vitreous, and lens of the eye, it is absorbed and attenuated by water, which is the main component of these tissues. The transmittance of light with respect to water is wavelength dependent, and it has a high transmittance in the wavelength range of approximately 200 nm to 900 nm. On the other hand, the human eye has a sensitivity characteristic for each wavelength of light called spectral luminous efficiency, which senses light from approximately 360 nm to 800 nm. For more stable measurements, it is suggested to avoid the above sensitivity range because it prevents miosis of the human eye. In addition, considering the absorption of water as well, light in the range of approximately 800 nm to 900 nm is desirable.

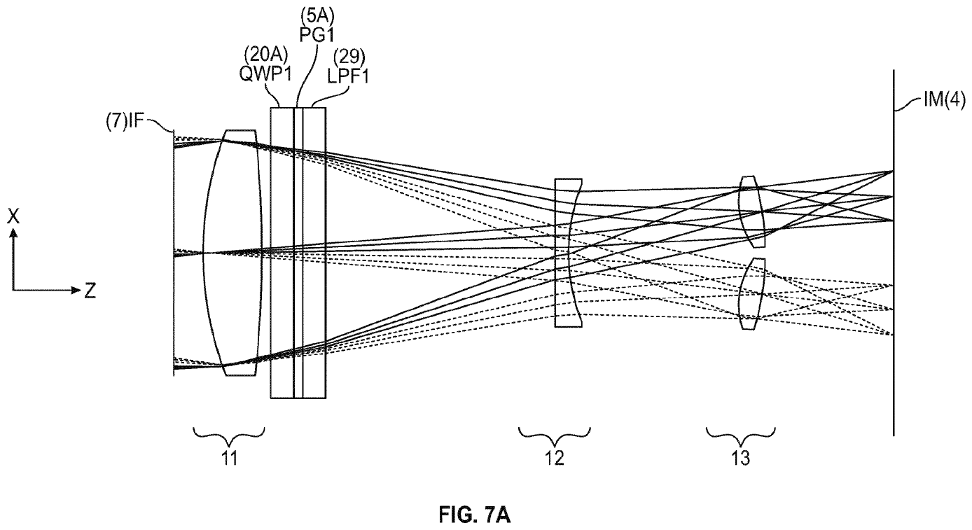

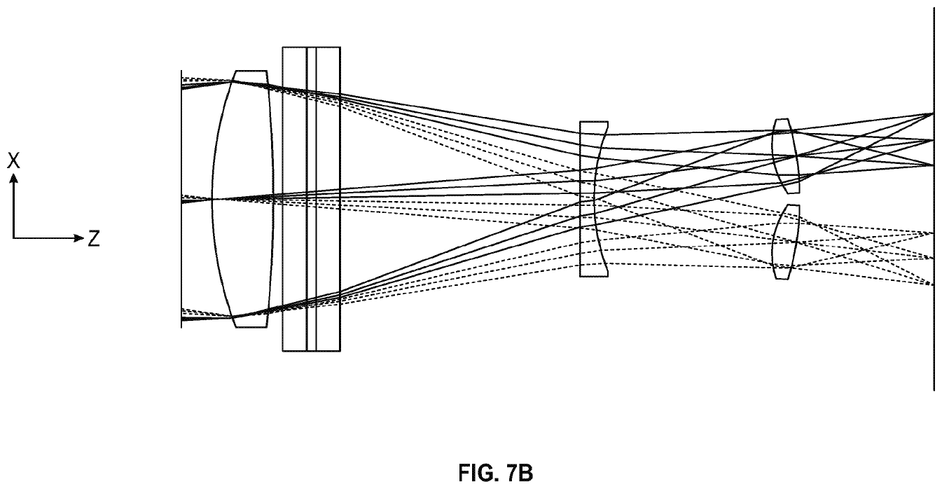

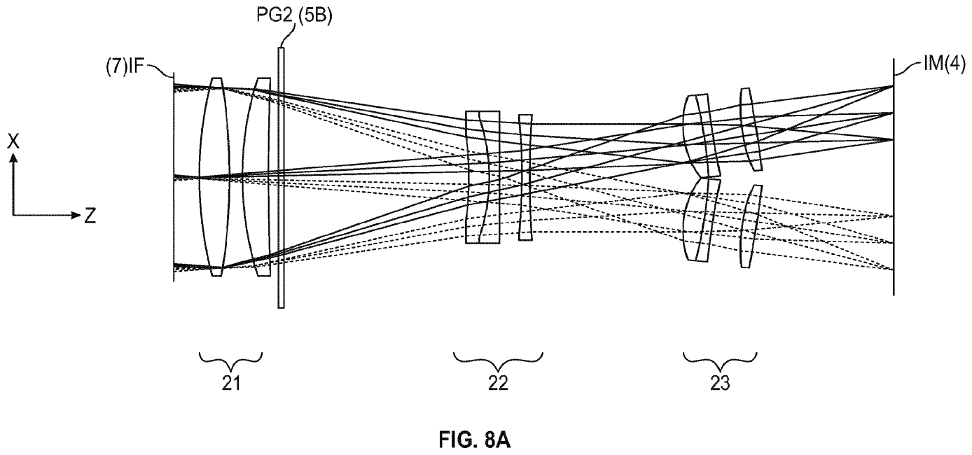

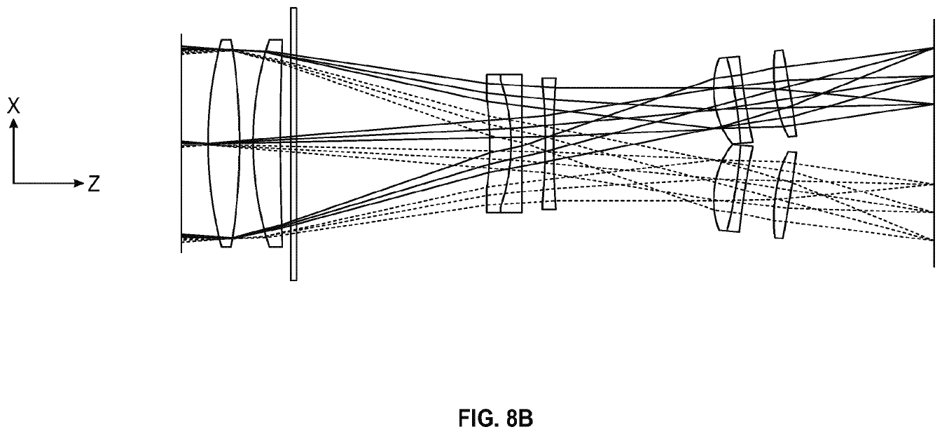

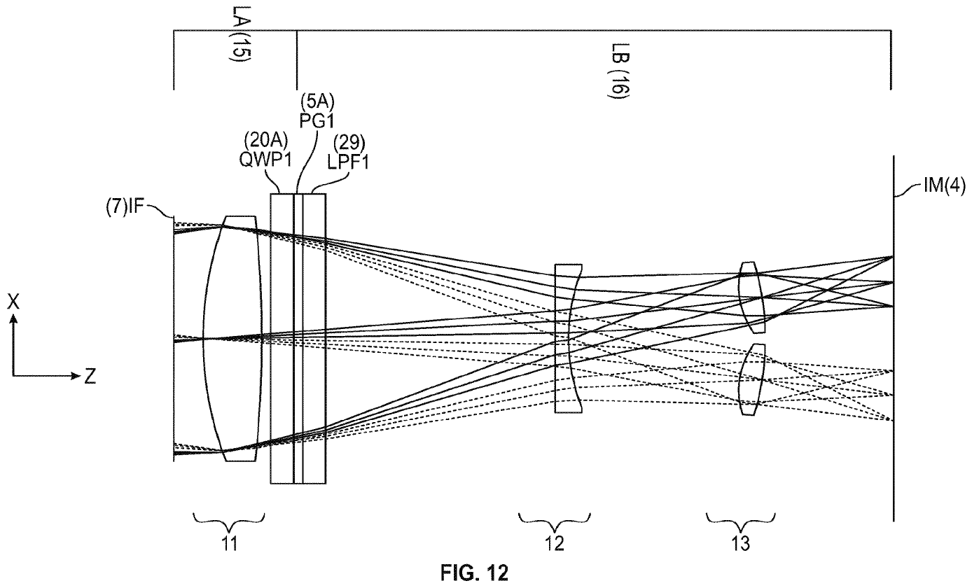

The reflected light from the retina passes through the half-mirror HM (26) and is guided to the imaging device by the imaging optical system. FIG. 6A shows the diffraction direction of the polarization-selective diffractive optical element (5) on the XZ plane and FIG. 6B on the YZ plane. FIG. 7A-B is an enlarged view of the imaging optical system of Example 1. FIG. 12 is an additional image of FIG. 7A in which LA (15) and LB (16) have been depicted. In addition, FIGS. 8A-B and 9A-B are enlarged views of the imaging optical system of Examples 2 and 3. Examples 2 and 3 omit the illuminator PJ (28), the eyes EY1 (9A) and EY2 (9B), and the half mirror HM (26). 11, 21, and 31 denote the first optical system, PG1 (5A), PG2 (5B), and PG3 (5C) denote the polarization-selective diffractive optical element, 12, 22, and 32 denote the second optical system, and 13, 23, and 33 denote the array optical system, respectively. In this embodiment, the array optical system 13, 23 and 33 have an aperture function. And, QWP1 (20A) indicates a quarter-wave plate and LPF1 (29) a long-pass filter. By arranging the long-pass filter LPF1 (29) on the optical path, stray light caused by external light etc. can be suppressed.

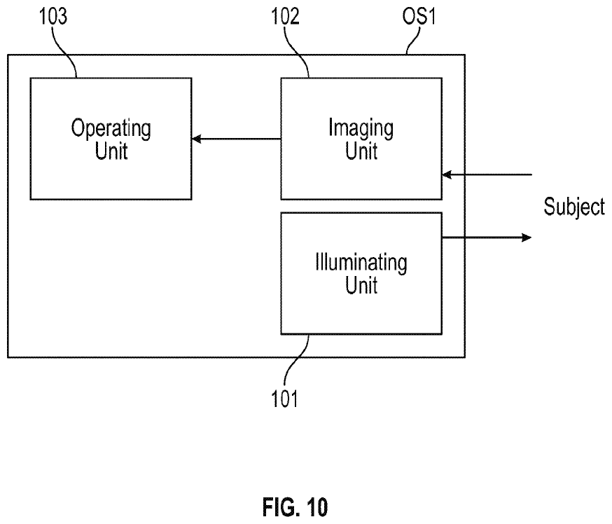

Next, a measurement device (vision fixation measurement device) OS1 will be described with reference to FIG. 10. FIG. 10 is a block diagram of the measurement device OS1. The measurement device OS1 can image the reflected light from the retina of the subject's eye by the imaging unit (102), identify the fovea of the retina from the change in polarization between the incident light to the retina of the subject's eye and the reflected light from the retina by the operation unit (103), and measure where the subject is looking (vision fixation state).

The measurement device OS1 has an illumination unit (101), an imaging unit (102), and an operation unit (103). The illumination unit (101) has a display surface or a light source such as an LED, a laser diode, etc., and guides the light toward the subject. The imaging unit (102) has an imaging optical system and an imaging device. The imaging unit (102) images the reflected light reflected from the retina (fundus) of the subject's eye. The imaging unit (102) also images information in relation to the polarization of the incident light to the retina and the reflected light from the retina. Moreover, the imaging unit (102) simultaneously photographs reflected light from the left and right eyes of the subject. The operation unit (103) is equipped with a means for measuring the vision fixation state of the subject based on information reflected from the retina of both eyes.

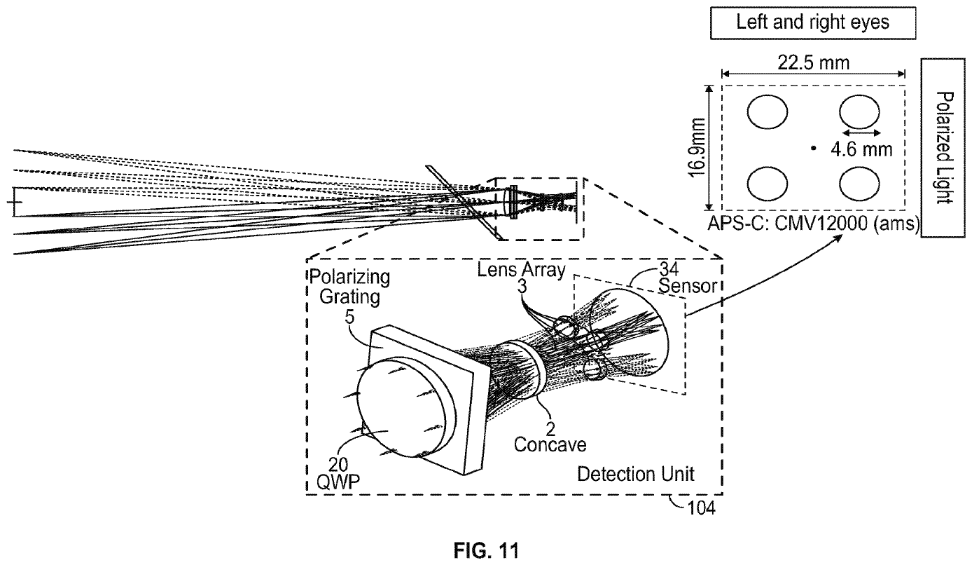

FIG. 11 depicts another embodiment of the measurement device provided in FIG. 10, including detection unit (104). In FIG. 11, vision fixation state from both left and right eyes of a subject can be detected. The device of FIG. 11 can comprise a circularly polarized light source, a light branching element such as a polarization-selective diffractive optical element PG (5), lenses which are arranged in order: convex lens (1), concave lens (2), and convex lens (3) (which correspond for ex., to reference numbers 11, 21 and 31 or 12, 22, and 32, or 31, 32 and 33), which form an intermediate image after the circularly polarized light is branched by the light branching element. Quarter wave plate QWP (20) and polarization gratings PG (5) can be located near a first lens placed by the formed intermediate image (IF) (not pictured). The last convex lens comprises four convex arrays (3) and the convex arrays (3) form an image of a retina of the eyes being diagnosed which can then be captured by an imaging system having sensor (34).

### EXAMPLES

Numerical data corresponding to Examples 1 to 3 are shown below. R denotes the radius of curvature of the i plane of the reading optical system, D denotes the spacing between the i plane and the i+1 plane of the reading optical system, and Na denotes the refractive index between the i plane and the i+1 plane in the d line. The radius of curvature R shall be positive if it is convex toward the intermediate image and negative if it is concave. The spacing D shall be positive in the direction toward the contracted conjugate plane side. Furthermore, v a is obtained by the following formula:

$${v}_{d} = \frac{{N}_{d} - 1}{{N}_{F} - {N}_{C}}$$

Here, N_{F} denotes the refraction index in the F line and Nc denotes the refraction index in the C line. In addition, the reference wavelength of each example shall be 830 nm. For the array optical system (13, 23, and 33), the virtual point determined by the spacing from the most imaging-side plane of the second optical system (12, 22, and 23) is set as the origin of the array optical system, and is shown in the tables as the eccentricity from that origin, respectively. The positions of the subsequent planes are arranged based on the local coordinate system formed by the most object-side plane of the array optical system. In addition, the spacing up to the imaging plane IM (4) indicated by (*) is defined as the distance from the plane of plane number (Surf No.) 2, respectively. For example, in Example 1, the imaging plane IM (4) is 60.00 mm away from the plane of plane number 2. And for example, in Example 2, the imaging plane IM (4) is 67.50 mm away from the plane of plane number 2. And for example, in Example 3, the imaging plane IM (4) is 54.00 mm away from the plane of plane number 2.

The diffraction planes of the polarization-selective diffractive optical element are arranged on the object-side plane, respectively. Each diffraction pitch and direction is shown below.

Although the optical surface in the Examples 1 to 3 is composed of a spherical surface of rotational symmetry, a rotationally symmetric aspherical surface, an anamorphic surface, or a free-form surface may be adopted as needed. In addition, cover glass, dustproof glass, etc. may be arranged on the optical path. Moreover, folding, etc. by a reflecting surface may be adopted depending on the device layout.

The following abbreviations will be used in presenting the data in the following examples:
R: Radius of curvature
D: Distance between surfaces
Nd: Refractive Index at d line
v_{d}: Abbe Number at d line
IF: Intermediate image
QWP: Quarter Wave Plate
PG: Polarization Gratings
LPF: Long Pass Filter
IM: Image plane

### Example 1

The configuration provided in FIG. 7A-B was used for this Example. Ref. 11, 12 and 13 as depicted in FIG. 7A are referenced in column 2 of Table 1 below.

TABLE 1

| Surf No. | | R | D | N | v |
|---|---|---|---|---|---|
| 1 | IF | 0.00 | 2.50 | 1.000 | |
| 2 | 11 | 35.00 | 5.00 | 1.760 | 49.60 |
| 3 | | −130.00 | 1.00 | 1.000 | |
| 4 | QWP1 | 0.00 | 2.00 | 1.516 | 58.59 |
| 5 | | 0.00 | 0.10 | 1.000 | |
| 6 | PG1 | 0.00 | 0.45 | 1.520 | 55.00 |
| 7 | | 0.00 | 0.10 | 1.000 | |
| 8 | LPF1 | 0.00 | 2.20 | 1.510 | 64.17 |
| 9 | | 0.00 | 20.00 | 1.000 | |
| 10 | 12 | 0.00 | 1.00 | 1.687 | 55.53 |
| 11 | | 19.91 | 15.00 | 1.000 | |
| 12 | 13 | 10.12 | 2.00 | 1.760 | 49.60 |
| 13 | | −23.23 | 0.00 | 1.000 | |
| 14 | | 0.00 | 60.00* | 1.000 | |
| 15 | IM | 0.00 | 0.00 | | |

The eccentricity amount of the array optical system 13 is shown below.

TABLE 2

| | | | X Axis | Y Axis |
|---|---|---|---|---|
| | X Shift | Y Shift | Rotation | Rotation |
| 1 | 3.7 | 3.4 | 9.74 | −9.67 |
| 2 | 3.7 | −3.4 | −9.74 | −9.67 |
| 3 | −3.7 | 3.4 | 9.74 | 9.67 |
| 4 | −3.7 | −3.4 | −9.74 | 9.67 |

polarization-selective diffractive optical element: Sixth plane
Diffraction pitch: 11.9 μm
Diffraction direction: Y

The image plane IM is located 60.00 (mm) away from the Surf No. 2. D of Surf No. 12 represents a thickness of the lens 13.

The lens 13 has a function of aperture.

Each of the lenses 13 is shifted and rotated from the position in the Table 1 according to the eccentricity in Table 2. The center position of the rotation is the position of the surface vertex of the object side surface of the lens 13 before the shift and the rotation.

### Example 2

The configuration provided in FIG. 8A-B was used for this Example. Ref. 21, 22 and 23 as depicted in FIG. 8A are referenced in column 2 of Table 3 below.

TABLE 3

| Surf No. | | R | D | N | v |
|---|---|---|---|---|---|
| 1 | IF | 0.00 | 2.50 | 1.000 | |
| 2 | 21 | 38.00 | 3.00 | 1.510 | 64.14 |
| 3 | | −80.00 | 1.25 | 1.000 | |
| 4 | | 42.30 | 2.50 | 1.510 | 64.14 |
| 5 | | 200.00 | 1.00 | 1.000 | |
| 6 | PG2 | 0.00 | 0.45 | 1.453 | 67.80 |
| 7 | | 0.00 | 18.00 | 1.000 | |
| 8 | 22 | −70.00 | 2.00 | 1.643 | 58.55 |
| 9 | | −22.00 | 0.85 | 1.626 | 34.47 |
| 10 | | 190.00 | 2.30 | 1.000 | |
| 11 | | −100.00 | 0.80 | 1.626 | 34.47 |
| 12 | | 70.00 | 15.00 | 1.000 | |
| 13 | 23 | 9.80 | 2.00 | 1.595 | 60.64 |
| 14 | | −54.50 | 0.80 | 1.741 | 26.52 |
| 15 | | 110.00 | 3.00 | 1.000 | |
| 16 | | 21.00 | 1.30 | 1.760 | 49.60 |
| 17 | | 0.00 | 0.00 | 1.000 | |
| 18 | | 0.00 | 67.50* | 1.000 | |
| 19 | IM | 0.00 | 0.00 | | |

The eccentricity amount of the array optical system 23 is shown below.

TABLE 4

| | | | X Axis | Y Axis |
|---|---|---|---|---|
| | X Shift | Y Shift | Rotation | Rotation |
| 1 | 4.0 | 4.0 | 8.50 | −8.50 |
| 2 | 4.0 | −4.0 | −8.50 | −8.50 |
| 3 | −4.0 | 4.0 | 8.50 | 8.50 |
| 4 | −4.0 | −4.0 | −8.50 | 8.50 |

polarization-selective diffractive optical element: Sixth plane
Diffraction pitch: 11.9 μm
Diffraction direction: Y

### Example 3

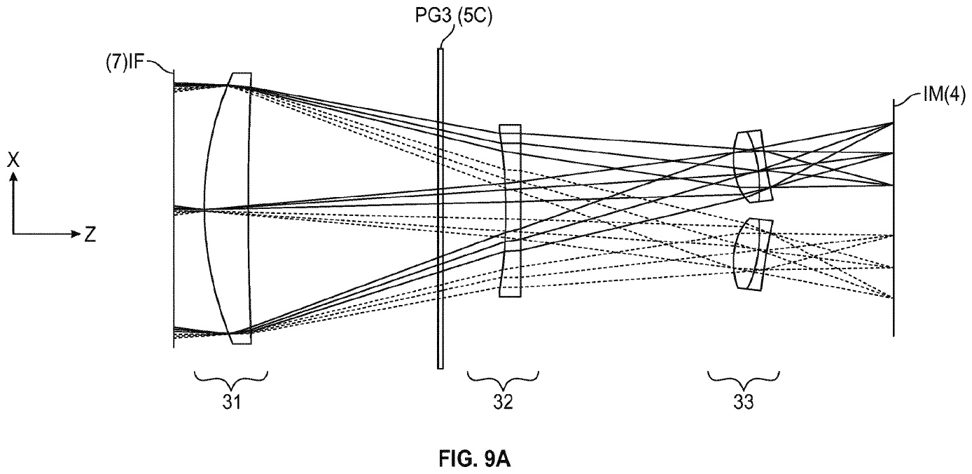

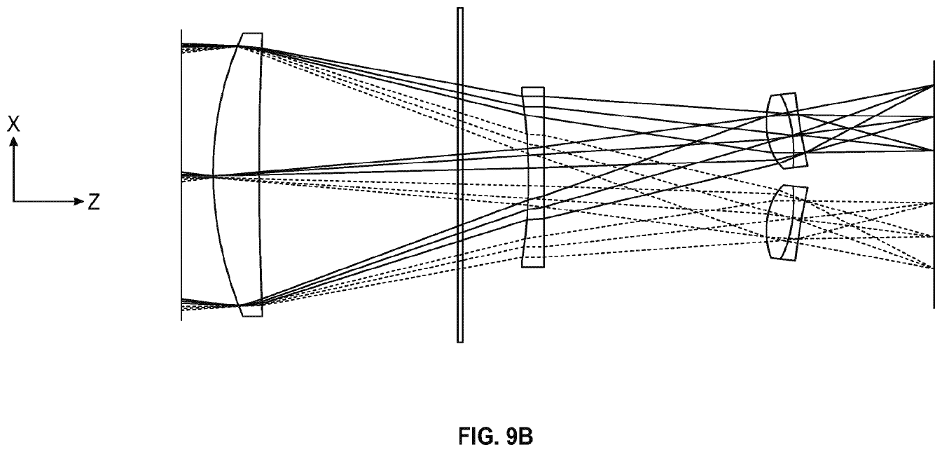

The configuration provided in FIG. 9A-B was used for this Example. Ref. 31, 32 and 33 as depicted in FIG. 9A are referenced in column 2 of Table 5 below.

TABLE 5

| Surf No. | | R | D | N | v |
|---|---|---|---|---|---|
| 1 | IF | 0.00 | 3.00 | 1.000 | |
| 2 | 31 | 29.00 | 3.50 | 1.822 | 23.78 |
| 3 | | 0.00 | 15.00 | 1.000 | |
| 4 | PG3 | 0.00 | 0.45 | 1.520 | 55.00 |
| 5 | | 0.00 | 5.00 | 1.000 | |
| 6 | 32 | −40.00 | 1.00 | 1.687 | 55.53 |
| 7 | | 0.00 | 17.00 | 1.000 | |
| 8 | 33 | 7.00 | 2.00 | 1.719 | 54.68 |
| 9 | | −15.00 | 0.70 | 1.626 | 34.47 |
| 10 | | 0.00 | 0.00 | 1.000 | |
| 11 | | 0.00 | 54.00* | 1.000 | |
| 12 | IM | 0.00 | 0.00 | | |

The eccentricity amount of the array optical system 33 is shown below.

TABLE 6

| | | | X Axis | Y Axis |
|---|---|---|---|---|
| | X Shift | Y Shift | Rotation | Rotation |
| 1 | 3.4 | 3.1 | 10.00 | −9.50 |
| 2 | 3.4 | −3.1 | −10.00 | −9.50 |
| 3 | −3.4 | 3.1 | 10.00 | 9.50 |
| 4 | −3.4 | −3.1 | −10.00 | 9.50 |

Polarized diffractometer: Sixth plane
Diffraction pitch: 6.8 μm
Diffraction direction: Y

The values of the conditional expressions in the imaging optical system of numerical examples 1 to 3 are shown in Table 7 below.

TABLE 7

| | Example 1 | Example 2 | Example 3 |
|---|---|---|---|
| LA | 8.50 | 10.25 | 21.50 |
| LB | 54.00 | 59.75 | 35.50 |

Although the embodiments of the present disclosure has been described above, the present disclosure is not limited to these embodiments, and various modifications and changes may be made within the scope of the abstract.

In referring to the description, specific details are set forth in order to provide a thorough understanding of the examples disclosed. In other instances, well-known methods, procedures, components and circuits have not been described in detail as not to unnecessarily lengthen the present disclosure.

It should be understood that if an element or part is referred herein as being “on”, “against”, “connected to”, or “coupled to” another element or part, then it can be directly on, against, connected or coupled to the other element or part, or intervening elements or parts may be present. In contrast, if an element is referred to as being “directly on”, “directly connected to”, or “directly coupled to” another element or part, then there are no intervening elements or parts present. When used, term “and/or”, includes any and all combinations of one or more of the associated listed items, if so provided.

Spatially relative terms, such as “under” “beneath”, “below”, “lower”, “above”, “upper”, “proximal”, “distal”, and the like, may be used herein for ease of description to describe one element or feature's relationship to another element(s) or feature(s) as illustrated in the various figures. It should be understood, however, that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, a relative spatial term such as “below” can encompass both an orientation of above and below. The device may be otherwise oriented (rotated 90 degrees or at other orientations) and the spatially relative descriptors used herein are to be interpreted accordingly. Similarly, the relative spatial terms “proximal” and “distal” may also be interchangeable, where applicable.

The term “about,” as used herein means, for example, within 10%, within 5%, or less. In some embodiments, the term “about” may mean within measurement error.

The terms first, second, third, etc. may be used herein to describe various elements, components, regions, parts and/or sections. It should be understood that these elements, components, regions, parts and/or sections should not be limited by these terms. These terms have been used only to distinguish one element, component, region, part, or section from another region, part, or section. Thus, a first element, component, region, part, or section discussed below could be termed a second element, component, region, part, or section without departing from the teachings herein.

The terminology used herein is for the purpose of describing particular embodiments only and is not intended to be limiting. The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “includes”, “including,” and “containing” are to be construed as open-ended terms (i.e., meaning “including, but not limited to,”) unless otherwise noted. Specifically, these terms, when used in the present specification, specify the presence of stated features, integers, steps, operations, elements, and/or components, but do not preclude the presence or addition of one or more other features, integers, steps, operations, elements, components, and/or groups thereof not explicitly stated. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Particular terms and abbreviations used throughout the disclosure include:

| | |
|---|---|
| 102 | Imaging unit |
| PG | polarization-selective diffractive optical element |
| AL | Array optical system |
| LE1 | First optical system |

It will be appreciated that the methods and compositions of the instant disclosure can be incorporated in the form of a variety of embodiments, only a few of which are disclosed herein. Variations of those embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed is:

1. An imaging optical system for guiding reflected light from the retina of the eye to an imaging device,
wherein the imaging optical system forms an intermediate image conjugate to the retina and the imaging plane of the imaging device, and
wherein the imaging optical system includes a first optical system, a polarization-selective diffractive optical element, and an array optical system in order along an optical path direction from the retina, and
separates the polarization state of the reflected light by the polarization-selective diffractive optical element.

2. The imaging optical system according to claim 1, wherein LA≤LB, if LA is the distance in the optical path direction from the intermediate image to the diffraction plane of the polarization-selective diffractive optical element, and LB is the distance in the optical path direction from the diffraction plane of the polarization-selective diffractive optical element to the imaging plane.

3. The imaging optical system according to claim 1, wherein the imaging optical system comprises a second optical system between the polarization-selective diffractive optical element and the array optical system,
the first optical system having positive power, the second optical system having negative power, and the array optical system having positive power.

4. The imaging optical system according to claim 1, wherein the imaging optical system simultaneously acquires a retinal image of the left eyeball and a retinal image of the right eyeball.

5. The imaging optical system according to claim 1, wherein each optical axis formed by the array optical system is inclined and eccentric with respect to the first optical axis formed by the first and second optical systems.

6. The imaging optical system according to claim 1, having a light-shielding part between the second optical system and the array optical system.

7. The imaging optical system according to claim 1, wherein the array optical system comprises: a first array lens for imaging reflected light that contains the first polarization information of the right eye among the retinae onto the imaging surface; a second array lens for imaging reflected light that contains the first polarization information of the left eye onto the imaging surface; a third array lens for imaging reflected light that contains the second polarization information of the right eye that is perpendicular to the first polarization information onto the imaging surface; and a fourth array lens for imaging reflected light that contains the second polarization information of the left eye onto the imaging surface.

8. The imaging optical system according to claim 1, comprising a ¼ wavelength plate on the intermediate image side of the polarization-selective diffractive optical element.

9. A measurement device comprising the imaging optical system indicated in claim 1, and one imaging device on the image plane of the optical system, and measuring the vision fixation state of a subject based on signal information of the retina acquired by the imaging device.

10. The measurement device according to claim 9, wherein the measuring device comprises an illuminator, and the illuminator forms an image conjugate to the intermediate image.

11. The measuring device according to claim 9, wherein the wavelength of light illuminated by the illuminator is 800~900 nm.
